(19) United States
(12) Patent Application Publication
(10) Pub. No.: US 2021/0228113 A1
(43) Pub. Date: Jul. 29, 2021

(54) Component Concentration Measurement Device

(71) Applicant: Nippon Telegraph and Telephone Corporation, Tokyo (JP)

(72) Inventors: Masahito Nakamura, Tokyo (JP); Yujiro Tanaka, Tokyo (JP); Michiko Seyama, Tokyo (JP); Katsuhiro Ajito, Tokyo (JP); Daichi Matsunaga, Tokyo (JP)

(21) Appl. No.: 15/734,771

(22) PCT Filed: May 17, 2019
(86) PCT No.: PCT/JP2019/019738

§ 371 Date: Dec. 3, 2020

(30) Foreign Application Priority Data
Jun. 7, 2018 (JP) 2018-109400

(51) Int. Cl. A61B 5/145; A61B 5/00

(52) U.S. Cl. CPC A61B 5/14532; A61B 5/0095

(57) ABSTRACT

A component concentration measurement device includes a light application unit (101) that applies pulsed beam light (121) of a wavelength that is absorbed by glucose to a site of measurement (151), and a detection unit (102) that detects a photoacoustic signal which is generated at the site of measurement (151) where the beam light (121) emitted from the light application unit (101) has been applied. The component concentration measurement device also includes a moisture measurement unit (103) that measures an amount of moisture in skin at the site of measurement (151), and a correction unit (104) that corrects an acoustic signal detected by the detection unit (102) with the amount of moisture measured by the moisture measurement unit (103).

### CROSS-REFERENCE TO RELATED APPLICATIONS

This application is a national phase entry of PCT Application No. PCT/JP2019/019738, filed on May 17, 2019, which claims priority to Japanese Application No. 2018-109400, filed on Jun. 7, 2018, which applications are hereby incorporated herein by reference.

### TECHNICAL FIELD

The present invention relates to a component concentration measurement device for non-invasively measuring glucose concentration.

### BACKGROUND

In terms of determining a dose of insulin for a diabetes patient or preventing diabetes, it is important to know (measure) blood sugar level. The blood sugar level is the concentration of glucose in blood, and as a way of measuring this kind of component concentration, a photoacoustic method is well known (see Patent Literature 1).

When a certain amount of light (an electromagnetic wave) is applied to a living body, the applied light is absorbed by molecules contained in the living body. As a result, target molecules for measurement in a portion applied with the light are locally heated to expand and generate a sound wave. The pressure of the sound wave depends on the amount of molecules that absorb the light. The photoacoustic method measures this sound wave to measure the amount of molecules in the living body. A sound wave is a pressure wave that propagates within a living body and has a property of being resistant to scattering compared to an electromagnetic wave; the photoacoustic method can be regarded to be a suitable way for measuring blood components in a living body.

Measurement by the photoacoustic method enables continuous monitoring of the glucose concentration in blood. In addition, measurement with the photoacoustic method does not require blood sample and causes no discomfort in a subject of measurement.

### CITATION LIST

### Patent Literature

Patent Literature 1: Japanese Patent Laid-Open No. 2010-104858

### SUMMARY

### Technical Problem

A site on a human body that is subjected to this type of measurement (e.g., skin) changes in amount of moisture over time. For example, the amount of moisture in skin changes over a certain time period after eating or drinking. When the amount of moisture at the site of measurement thus changes, however, a measurement result of glucose measurement in a human body by the photoacoustic method will change. As the measurement result changes due to such a change in amount of moisture, it can happen that concentrations are actually the same when results that were measured at different times are different or that concentrations are actually different when results that were measured at different times are the same, which hinders an accurate measurement.

In order to solve such a drawback, an object of embodiments of the present invention is to suppress decrease in measurement accuracy that is caused by a change in moisture in a human body when glucose in a human body is measured by the photoacoustic method.

### Means for Solving the Problem

A component concentration measurement device according to embodiments of the present invention includes: a light application unit that applies beam light of a wavelength that is absorbed by glucose to a site of measurement; a detection unit that detects a photoacoustic signal which is generated at the site of measurement where the beam light emitted from the light application unit has been applied; a moisture measurement unit that measures an amount of moisture in skin at the site of measurement; and a correction unit that corrects an acoustic signal detected by the detection unit with the amount of moisture measured by the moisture measurement unit.

The component concentration measurement device may include a plurality of moisture measurement units, and the correction unit may correct the acoustic signal detected by the detection unit with an average of a plurality of amounts of moisture measured by the plurality of moisture measurement units.

In the component concentration measurement device, the light application unit may include a light source unit that generates the beam light of a wavelength that is absorbed by glucose; and a pulse control unit that turns the beam light generated by the light source unit into pulsed light of a set pulse width.

### Effects of embodiments of the Invention

As described above, according to embodiments of the present invention, the amount of moisture in skin at the site of measurement is measured and an acoustic signal detected by the detection unit is corrected with the measured amount of moisture. Thus, it provides an advantageous effect of suppressing decrease in measurement accuracy that is caused by a change in moisture in a human body when glucose in a human body is measured by the photoacoustic method.

### BRIEF DESCRIPTION OF THE DRAWINGS

FIG. 1 is configuration diagram showing a configuration of a component concentration measurement device in an embodiment of the present invention.

FIG. 2 is a configuration diagram showing a more detailed configuration of a light source unit 105 and a detection unit 102 in an embodiment of the present invention.

FIG. 3 is a characteristic diagram showing the relationship between dielectric constant εE(t) and moisture content at a location of measurement.

FIG. 4 is a characteristic diagram showing an experiment result for a measurement of glucose concentration in a living body with the component concentration measurement device in an embodiment.

### DETAILED DESCRIPTION OF ILLUSTRATIVE EMBODIMENTS

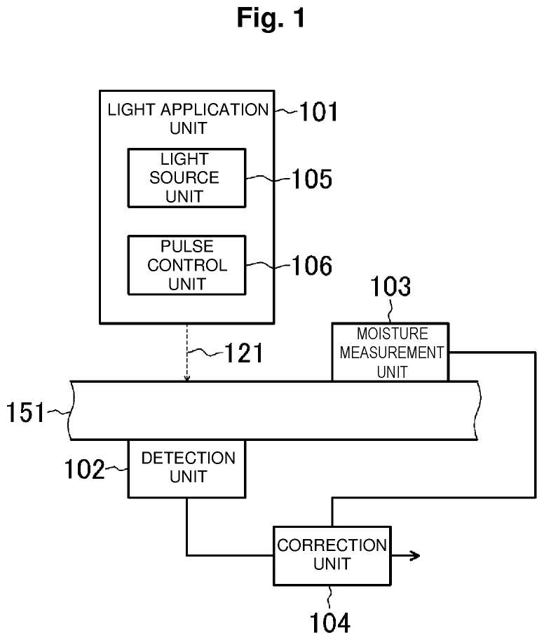

A component concentration measurement device according to an embodiment of the present invention is described below with reference to FIG. 1. The component concentration measurement device includes a light application unit 101 that applies pulsed beam light 121 of a wavelength that is absorbed by glucose to a site of measurement 151, and a detection unit 102 that detects a photoacoustic signal generated in the site of measurement 151 where the beam light 121 emitted from the light application unit 101 has been applied.

For example, the light application unit 101 includes a light source unit 105 that generates the beam light 121 of a wavelength that is absorbed by glucose, and a pulse control unit 106 that turns the beam light 121 generated by the light source into pulsed light of a set pulse width. Glucose exhibits absorbency in light wavelength bands around 1.6 μm and around 2.1 μm (see Patent Literature 1). The beam light 121 has a beam diameter of about 100 μm, for example.

The component concentration measurement device also includes a moisture measurement unit 103 that measures an amount of moisture in skin at the site of measurement 151, and a correction unit 104 that corrects an acoustic signal detected by the detection unit 102 with the amount of moisture measured by the moisture measurement unit 103.

The moisture measurement unit 103 can be a dermometry-based (impedance-based) skin moisture meter, a capacitive skin moisture meter, or a microwave-based skin moisture meter, for example. The moisture measurement unit 103 may be positioned near a location to be applied with the beam light 121, for example. Alternatively, multiple moisture measurement units 103 may be positioned so as to surround the location to be applied with the beam light 121 and an average of measurement results from them may be used as the amount of moisture. The site of measurement 151 is a portion of a human body, like a finger or an ear lobe, for example.

The correction unit 104 corrects an acoustic signal detected by the detection unit 102 with an amount of moisture which has been measured by the moisture measurement unit 103 within a preset time from the point when the detection unit detected the acoustic signal. For example, the acoustic signal detected by the detection unit 102 is corrected with the amount of moisture which was measured by the moisture measurement unit 103 at the point when the detection unit 102 detected the acoustic signal. For example, a state of temporal change in the amount of moisture at the site of measurement 151 is measured in advance to determine an amount of time that causes a change in the amount of moisture that needs correction, and the aforementioned preset time may be set based on the result.

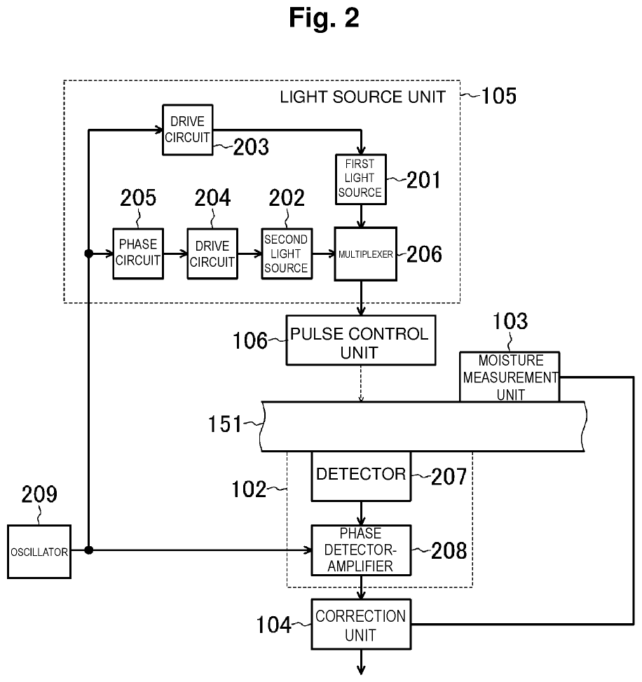

The light source unit 105 includes a first light source 201, a second light source 202, a drive circuit 203, a drive circuit 204, a phase circuit 205, a multiplexer 206, a detector 207, a phase detector-amplifier 208, and an oscillator 209 as shown in FIG. 2. The first light source 201, the second light source 202, the drive circuit 203, the drive circuit 204, the phase circuit 205, and the multiplexer 206 constitute the light source unit 105. The detector 207 and the phase detector-amplifier 208 constitute the detection unit 102.

The oscillator 209 is connected to each of the drive circuit 203, the phase circuit 205, and the phase detector-amplifier 208 via signal wires. The oscillator 209 sends a signal to each of the drive circuit 203, the phase circuit 205, and the phase detector-amplifier 208.

The drive circuit 203 receives the signal sent from the oscillator 209, and supplies driving electric power to the first light source 201, which is connected by a signal wire, to cause the first light source 201 to emit light. The first light source 201 is a semiconductor laser, for example.

The phase circuit 205 receives the signal sent from the oscillator 209, and sends a signal generated by giving a phase shift of 180 ° to the received signal to the drive circuit 204, which is connected by a signal wire.

The drive circuit 204 receives the signal sent from the phase circuit 205, and supplies driving electric power to the second light source 202, which is connected by a signal wire, to cause the second light source 202 to emit light. The second light source 202 is a semiconductor laser, for example.

The first light source 201 and the second light source 202 output light of different wavelengths from each other and direct their respective output light to the multiplexer 206 via light wave transmission means. For the first light source 201 and the second light source 202, the wavelength of light of one of them is set to a wavelength that is absorbed by glucose, while the wavelength of light of the other is set to a wavelength that is absorbed by water. Their respective wavelengths are also set such that degrees of their absorption will be equivalent.

The light output by the first light source 201 and the light output by the second light source 202 are multiplexed in the multiplexer 206 and are incident onto the pulse control unit 106 as one light beam. Upon incidence of the light beam, the pulse control unit 106 applies the incident light beam to the site of measurement 151 as pulsed light of a predetermined pulse width. Inside the site of measurement 151 thus applied with the pulsed light beam, a photoacoustic signal is generated.

The detector 207 detects the photoacoustic signal generated in the site of measurement 151, converts it into an electric signal, and sends it to the phase detector-amplifier 208, which is connected by a signal wire. The phase detector-amplifier 208 receives a synchronization signal necessary for synchronous detection sent from the oscillator 209, and also receives the electric signal proportional to the photoacoustic signal being sent from the detector 207, performs synchronous detection, amplification and filtering on it, and outputs an electric signal proportional to the photoacoustic signal.

The first light source 201 outputs light that has been intensity-modulated in synchronization with an oscillation frequency of the oscillator 209. In contrast, the second light source 202 outputs light that has been intensity-modulated with the oscillation frequency of the oscillator 209 and in synchronization with the signal that has gone through a phase shift of 180° in the phase circuit 205.

Here, since the intensity of the signal output by the phase detector-amplifier 208 is proportional to the amount by which the light output from each of the first light source 201 and the second light source 202 was absorbed by components (glucose, water) in the site of measurement 151, the intensity of the signal is proportional to the amounts of components in the site of measurement 151.

As mentioned above, the light output by the first light source 201 and the light output by the second light source 202 have been intensity-modulated with signals of the same frequency. Accordingly, there is no effect of unevenness in frequency characteristics of a measurement system, which is problematic in the case of intensity modulation with signals of multiple frequencies.

Meanwhile, non-linear dependence on absorption coefficient that exists in measured values of photoacoustic signals, which is problematic in measurements by the photoacoustic method, can be solved by performing measurements using light of multiple wavelengths that gives an equal absorption coefficient as described above (see Patent Literature 1).

As mentioned above, the intensity of the acoustic signal output by the detection unit 102 is corrected by the correction unit 104, and based on a corrected correction value, a component concentration derivation unit (not shown) determines the amount of glucose component in blood within the site of measurement 151.

Next, correction by the correction unit 104 of an acoustic signal detected by the detection unit 102 with an amount of moisture measured by the moisture measurement unit 103 is described.

In a one-dimensional system, a photoacoustic signal at time t for a substance having a certain concentration distribution is represented as Formula (1).

$$[ Formula 1 ]$$

$$P ( t ) = {\int }_{0}^{∞} β ( x , t ) exp ( - \frac{( 1 + j ) x}{{μ}_{s}} ) d x \qquad ( 1 )$$

In Formula (1), P is the output of the photoacoustic signal, β(x, t) is an absorption coefficient at depth x and at a given wavelength when a radiation end surface of the light source is defined as x=o, and μs is thermal diffusion length.

The value β(x, t) in Formula (1) changes either when a target component concentration c changes or when a moisture content w changes, so that when considering measurement of skin, β(x, t) would be shown by “β(x, t)=w(t)×{c(t)+cₑ(t)}. . . (2)”. The term cₑ(t) is absorption by components other than the target component.

As will be apparent from Formulas (1) and (2), an acoustic signal also changes when there is a change in the moisture content. Here, a measurement result with the moisture measurement unit 103 can be represented by a linear expression such as “ε(t)=w(t)×α×ε_{water} . . . (3)”. The value ε(t) is a dielectric constant as measured by the moisture measurement unit 103, ε_{water} is the dielectric constant of water, and α is an arbitrary coefficient.

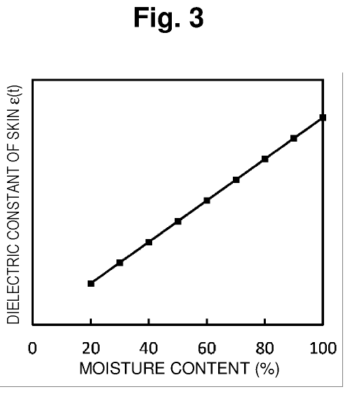

The relationship of Formula (3) (the relationship between the dielectric constant measured and the moisture content at the location of measurement) is indicated as in FIG. 3. Using an amount of change Δw(t) in the moisture content from time to, the output signal P(t) is corrected according to Formulas (4) and (5) shown below.

$$[ Formula 2 ]$$

$$\begin{matrix}P ( t ) ' = {\int }_{0}^{∞} β ( x , t ) / Δ w ( t ) \cdot exp ( - \frac{( 1 + J ) x}{{μ}_{s}} ) dx & ( 4 ) \\ Δ w ( t ) = \frac{ɛ ( t )}{ɛ ( t 0 )} = \frac{w ( f )}{w ( t 0 )} & ( 5 )\end{matrix}$$

The Δw(t) in Formula (4) for correction of moisture is measured at the same timing as the acquisition of the photoacoustic signal. Using such a correction, β(x, t)/Δw(t) will always be “β(x, t)/Δw(t)=w(to)·{c(t)+cₑ(t)}. . . (6)”, so that the effect of moisture content can be suppressed.

The correction described above enables an accurate measurement of a change in the concentration of the target component. Additionally, for dual wavelength photoacoustic signals, an increased accuracy of dual wavelength differential measurement can be expected by applying Formula (5) to each wavelength.

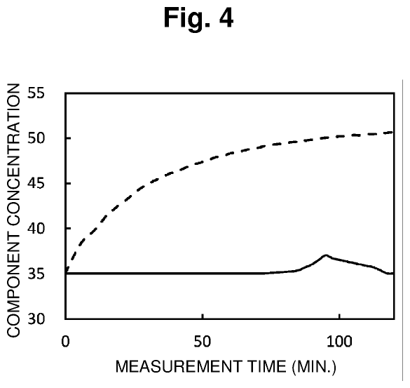

FIG. 4 shows an experiment result for a measurement of glucose concentration in a living body with the component concentration measurement device according to the above-described embodiment. In FIG. 4, the broken line indicates before correction and the solid line indicates after correction. As shown in FIG. 4, according to the embodiment, the effect of moisture content is suppressed, which enables an accurate measurement of the target component concentration.

As has been described above, according to the present invention, the amount of moisture in skin at the site of measurement is measured and an acoustic signal detected by the detection unit is corrected with the measured amount of moisture. Thus, it is possible to suppress decrease in measurement accuracy that is caused by a change in moisture in a human body when glucose in a human body is measured by the photoacoustic method.

It will be apparent that the present invention is not limited to the above-described embodiments but many variations and combinations may be made by ordinarily skilled persons in the art within the technical idea of the invention.

### REFERENCE SIGNS LIST

101 light application unit

102 detection unit

103 moisture measurement unit

104 correction unit

105 light source unit

106 pulse control unit

121 beam light

151 site of measurement.

1.-3. (canceled)

4. A component concentration measurement device comprising:
a light that applies beam light to a site of measurement, the beam light having a wavelength that is absorbed by glucose;
a detector that detects a photoacoustic signal which is generated at the site of measurement where the beam light has been applied;
a moisture measurer that measures an amount of moisture in skin at the site of measurement; and
a corrector that corrects an acoustic signal detected by the detector in accordance with the amount of moisture measured by the moisture measurer.

5. The component concentration measurement device according to claim 4, further comprising a plurality of the moisture measurers, wherein the moister measurer is one of the plurality of moister measurers, and wherein the corrector corrects the acoustic signal in accordance with an average of a plurality of amounts of moisture measured by the plurality of moisture measurers.

6. The component concentration measurement device according to claim 5, wherein the light comprises:
a light source that generates the beam light of the wavelength that is absorbed by glucose; and
a pulse controller that turns the beam light generated by the light source unit into pulsed light of a set pulse width.

7. The component concentration measurement device according to claim 4, wherein the light comprises:
a light source that generates the beam light of the wavelength that is absorbed by glucose; and
a pulse controller that turns the beam light generated by the light source unit into pulsed light of a set pulse width.

8. The component concentration measurement device according to claim 4, wherein the corrector corrects the acoustic signal in accordance with a dielectric constant of the amount of moisture measured by the moisture measurer.

9. A method comprising:
applying, by a light, beam light to a site of measurement, the beam light having a wavelength that is absorbed by glucose;
detecting, by a detector, a photoacoustic signal which is generated at the site of measurement where the beam light has been applied;
taking a moisture amount measurement of skin at the site of measurement; and
correcting an acoustic signal detected by the detector in accordance with the moisture amount measurement.

10. The method according to claim 9 further comprising taking a plurality of moisture amount measurements of the skin at the site of measurement, wherein the moisture amount measurement is one of the plurality of moisture amount measurements, and wherein correcting the acoustic signal comprises correcting the acoustic signal in accordance with an average of a plurality of moisture amount measurements.

11. The method according to claim 10, wherein the light comprises:
a light source that generates the beam light of the wavelength that is absorbed by glucose; and
a pulse controller that turns the beam light generated by the light source unit into pulsed light of a set pulse width.

12. The method according to claim 9, wherein the light comprises:
a light source that generates the beam light of the wavelength that is absorbed by glucose; and
a pulse controller that turns the beam light generated by the light source unit into pulsed light of a set pulse width.

13. The method according to claim 9, wherein moisture amount measurement is a dielectric constant of the amount of moisture of the skin at the site of measurement.
